(19) United States
(12) Patent Application Publication
(10) Pub. No.: US 2025/0213258 A1
(43) Pub. Date: Jul. 3, 2025

(54) MECHANICAL THROMBUS REMOVAL DEVICE

(71) Applicant: Althea Medical Ltd., Ariel (IL)

(72) Inventors: Raphael Benary, Tel Aviv (IL); Lia Ofek, Ramat Hasharon (IL); Or Chernin, Rosh Ha’Ayin (IL)

(73) Assignee: Althea Medical Ltd., Ariel (IL)

(21) Appl. No.: 19/085,230

(22) Filed: Mar. 20, 2025

Related U.S. Application Data
(63) Continuation-in-part of application No. PCT/IB2023/059277, filed on Sep. 19, 2023.
(60) Provisional application No. 63/491,120, filed on Mar. 20, 2023.
(60) Provisional application No. 63/376,617, filed on Sep. 22, 2022.

(51) Int. Cl. A61B 17/22; A61B 17/00; A61M 25/06

(52) U.S. Cl. CPC A61B 17/22; A61B 2017/00336; A61B 2017/22079; A61M 2025/0681

(57) ABSTRACT

A medical device includes a flexible cannula that includes an internal tube having a proximal end and a distal end and a series of slits cut along a length thereof between the proximal and the distal ends, and an outer jacket softer than the inner tube. Discrete portions of the outer tube are connected to the inner tube along a longitudinal axis of the cannula. Areas at which there is no connection between the outer jacket and the inner tube allow some relative movement between the outer jacket and the inner tube, resulting in increased flexibility of the cannula.

### FIELD OF THE INVENTION

The present invention generally relates to devices and methods for cardiac procedures, and particularly for thrombus removal.

### BACKGROUND OF THE INVENTION

Removal of obstructions, such as a thrombus, from vasculature is an important and life-saving technique.

Physicians often insert a cannula inserted into the vasculature to remove such obstructions. A negative pressure (vacuum) is generated at the proximal side of the cannula typically using a vacuum syringe, causing the obstruction to be aspirated into the syringe through the cannula. In many cases, the clot is too large and too well defined to fit through the cannula, causing the thrombus to remain at the distal end of the cannula instead of travelling back into the syringe through the cannula since it is only held in place by the force generated by the negative pressure within the cannula. The physician must then remove the cannula from the body carefully so as not to break vacuum and loose the clot. Thus, relying only on the force generated by vacuum may lead to the clot being released back into the blood vessel during extraction.

An improved device for removing a thrombus is thus needed.

### SUMMARY

The present invention seeks to provide an improved device for removing a thrombus, as is described more in detail hereinbelow.

There is thus provided in accordance with a non-limiting embodiment of the invention a medical device including a flexible cannula that includes an internal tube having a proximal end and a distal end and a series of slits cut along a length thereof between the proximal and the distal ends, and an outer jacket softer than the inner tube, wherein discrete portions of the outer tube are connected to the inner tube along a longitudinal axis of the cannula, and areas at which there is no connection between the outer jacket and the inner tube allow some relative movement between the outer jacket and the inner tube, resulting in increased flexibility of the cannula.

In accordance with a non-limiting embodiment of the invention the outer jacket is made of a meltable polymer, and the discrete portions of the outer jacket include melted portions that stick to an outside of the inner tube.

In accordance with a non-limiting embodiment of the invention the slits enabling the internal tube to bend in any direction by expansion and compression of the slits.

In accordance with a non-limiting embodiment of the invention the medical device further includes: a thrombus removal device that includes a distal removal element, coupled at a distal portion thereof to an inner shaft, and located within an outer shaft configured to guide the distal removal element towards a target site, wherein the distal removal element includes radial elements which are exposed by retracting the outer shaft, and wherein the distal removal element is opened by pulling the inner shaft relative to an intermediate shaft, thereby compressing the distal removal element and forcing the radial elements to open radially outward to an open position.

In accordance with a non-limiting embodiment of the invention a membrane covers the radial elements, and pulling the inner shaft causes a distal end of the membrane, which is connected to a radial plunger, to fold back onto itself and create a vacuum. The membrane may be a blood-impervious membrane.

In accordance with a non-limiting embodiment of the invention the inner shaft, the intermediate shaft and the outer shaft are concentric with each other.

In accordance with a non-limiting embodiment of the invention some of the radial arms have larger radial diameters in the open position than others of the radial arms.

In accordance with a non-limiting embodiment of the invention some of the radial arms have different sinusoidal or other wavy shapes than others of the radial arms.

In accordance with a non-limiting embodiment of the invention an adjustable seal valve is coupled to the flexible cannula, the adjustable seal valve including a first end which is fixed and a second end which is free to rotate relative the first end, and wherein a deformable internal sleeve is coupled to the first end and to the second end, and wherein radially placed indentations on the first end are configured to receive a protrusion placed on the second end so as to lock the second end into the first end and prevent rotation of the second end, and wherein rotation of the second end causes the deformable internal sleeve to deform and collapse inwardly in a radial direction so as to form a seal around an object of any diameter passing through the deformable internal sleeve.

### BRIEF DESCRIPTION OF DRAWINGS

The present invention will be understood and appreciated more fully from the following detailed description, taken in conjunction with the drawings in which:

FIG. 1 is a simplified illustration of a thrombus removal device, in accordance with a non-limiting embodiment of the invention.

FIG. 2 is a simplified illustration of a distal removal element being exposed by retracting an outer shaft of the device.

FIG. 3 is a simplified illustration representation of the thrombus removal device in place with the distal removal element fully exposed.

FIG. 4 is a simplified illustration of a mechanical removal element in an opened configuration.

FIG. 5 is a simplified illustration of a method used to remove the thrombus from the body lumen, in accordance with a non-limiting embodiment of the invention.

FIG. 6 is a simplified illustration of the mechanical removal element in its open position.

FIG. 7 is a simplified illustration of another thrombus removal device, in accordance with a non-limiting embodiment of the invention.

FIG. 8 is a simplified illustration of uncrimping the device of FIG. 7 from its crimped state.

FIG. 9 is a simplified illustration of the device of FIG. 7 fully uncrimped and free from the outer shaft.

FIG. 10 is a simplified illustration of the shafts of the device of FIG. 7, wherein an outer shaft can be moved to encompass the device and to radially compress radially compressible elements and a membrane.

FIG. 11 is a simplified illustration of the device of FIG. 7 prior to an inner tube being pulled back and causing vacuum locally.

FIG. 12 is a simplified illustration of the device of FIG. 7 with the membrane inverted inward once the inner shaft has been pulled back.

FIG. 13 is a simplified illustration of the device of FIG. 7 in an obstructed body lumen.

FIG. 14 is a simplified illustration of the obstruction swallowed by the device of FIG. 7 due to the vacuum generated by pulling back the inner shaft.

FIG. 15 is a simplified illustration of a thrombus removal device, in accordance with another non-limiting embodiment of the invention.

FIG. 16 is a simplified illustration of a cannula with a chamfered distal end, showing toothed rings with inwardly directional barbs.

FIG. 17 is a simplified illustration of insertion of the chamfered cannula into an obstructed blood vessel.

FIG. 18 is a simplified illustration of the cannula advanced up to the thrombus site.

FIG. 19 is a simplified illustration of the thrombus aspirated.

FIG. 19A is cutaway illustration showing how the thrombus gets snagged on the barbs of the toothed ring.

FIG. 20 is a simplified illustration of a thrombus removal device, in accordance with another non-limiting embodiment of the invention.

FIGS. 21 and 22 are simplified illustrations of an adjustable seal valve which can be attached to a proximal end of a cannula.

FIG. 23 is a simplified illustration of the adjustable valve with an inner tube deformed to an intermediate level to seal over a therapeutic tool.

FIG. 24 is a simplified illustration of a flexible cannula constructed by combining an internal tube having a proximal end and a distal end and a series of slits cut along is length.

FIG. 25 is a simplified illustration of gaps in the connection zones between the inner tube and the outer jacket.

FIG. 26 is a simplified illustration of slits that decrease wall friction in the inner tube.

FIG. 27 is a simplified illustration of a dual introducer sheath, in accordance with a non-limiting embodiment of the invention.

FIG. 28 is a simplified illustration of the dual sheath device showing the orientation of shafts and hubs prior to insertion into a blood vessel.

FIG. 29 is a simplified illustration of the outer sheath as it is pulled back relative to inner sheath so that hubs are connected.

FIG. 30 is a simplified illustration of the hubs in their locked position.

FIG. 31 is a simplified illustration of the inner sheath as it is removed from the outer sheath.

FIGS. 32A and 32B are simplified illustrations of another version of the embodiments of FIGS. 21, 22 and 23, in which a spring is coupled between the rotating collar and a portion of the body of valve.

### DETAILED DESCRIPTION

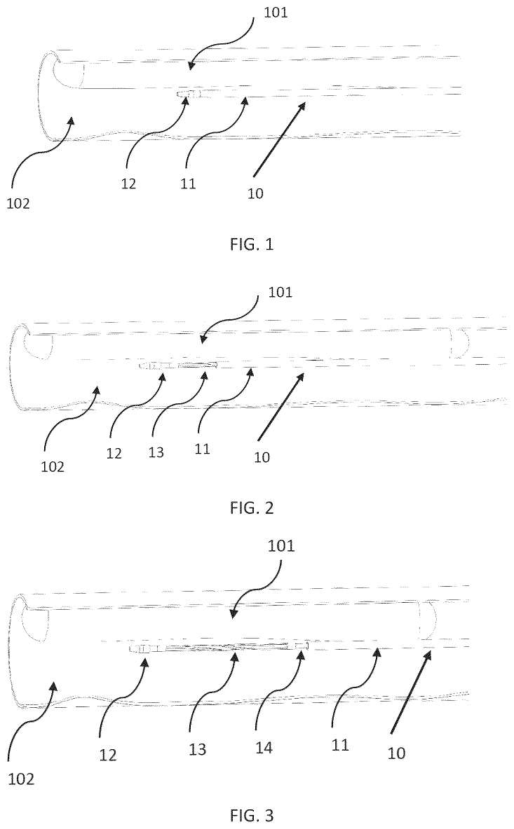

FIG. 1 illustrates a thrombus removal device 10 including a mechanical removal element (not shown in FIG. 1) which is attached to an inner guidewire shaft 12 (by its distal end) and is located within an outer shaft 11 which can enable it to be guided towards the target site. Device 10 may be inserted into a body lumen 101 using standard percutaneous techniques.

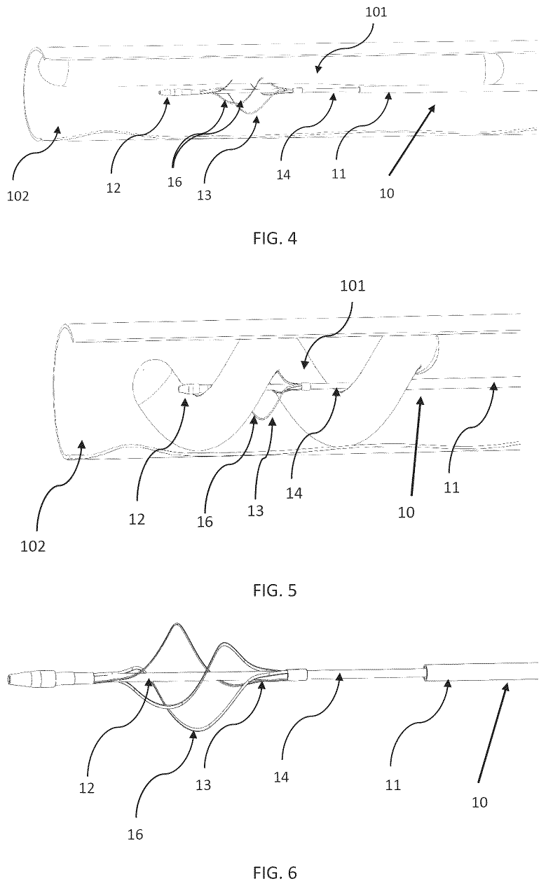

FIG. 2 illustrates the distal removal element 13 being exposed by retracting the outer shaft 11. This may be done once the distal end of device 10 has reached the thrombus 101. FIG. 3 illustrates the thrombus removal device 10 in place with the distal removal element 13 fully exposed.

FIG. 4 shows the mechanical removal element 13 in an opened configuration. In one embodiment of the present invention, a helical design is shown in which mechanical removal element 13 includes slender flexible radial arms 16 (such as wires or braids, which may be made of nitinol, stainless steel or other suitable materials) which are formed as a helix or arcuately-shaped wrappings of wire. The element 13 is opened by pulling the inner shaft 12 to which the distal end of element 13 is connected against a medial shaft 14 thus compressing it and forcing the radial arms 16 to open radially outward. This is done using a control handle (not shown). The physician can dial in several opening diameters depending on the patient's anatomy.

FIG. 5 is a schematic representation of the method used to remove the thrombus from the body lumen. Following the radial opening of the distal removal element arms 16, the physician uses the control handle to rotate inner shaft 12 and thereby rotate distal removal element 13. This action twists the thrombus 101, thus breaking its adherence to the arterial wall and causing the thrombus to twist around the device 10. The thrombus can then be pulled back and removed.

FIG. 6 illustrates the mechanical removal element 13 in its open position. Inner shaft 12 in its pulled back position, with element 13 pressed against it and the medial shaft 14. As a result, distal removal element radial arms 16 are open radially. Outer shaft 11 is shown its retracted state. In other embodiments, device 10 may have less than three arms 16 such as one arm, or more than three arms. It is noted that some radial arms 16 may have larger radial diameters in the open position than other radial arms 16. It is also noted that some radial arms 16 may have different sinusoidal or other wavy shapes than other radial arms 16.

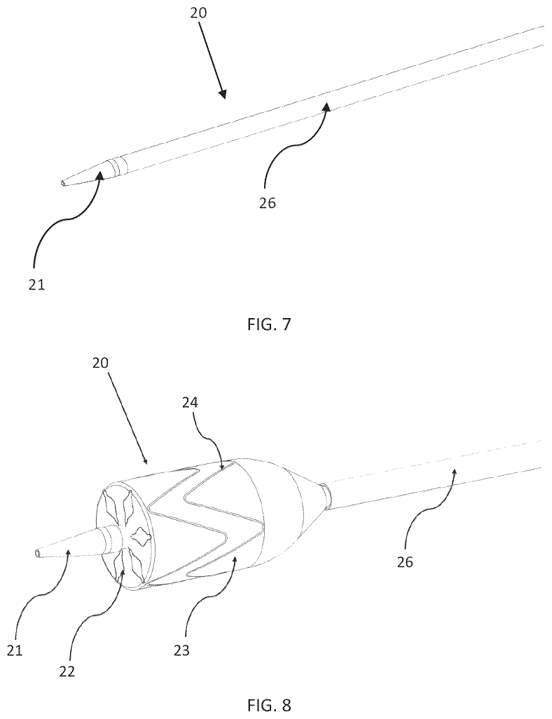

FIG. 7 illustrates another thrombus removal device 20 which may be inserted into a body lumen using standard percutaneous techniques. The device is shown in its fully crimped state. Outer shaft 26 is used to maintain the various elements of device 20 in their crimped state so as to allow for percutaneous delivery. Tip 21 ensures that the distal end of the device is atraumatic.

FIG. 8 illustrates the step of deploying device 20 from its crimped state. Elements 24 are self-expanding radial structures (made of self-expanding materials, such as but not limited to, superelastic alloys, such as nitinol) which can move from a crimped to a non-crimped state. A membrane 23 which is impervious to blood covers self-expanding radial elements 24. Membrane 23 extends from the proximal end of the device to its distal end. A self-expanding plunger element 22 is also covered by the membrane.

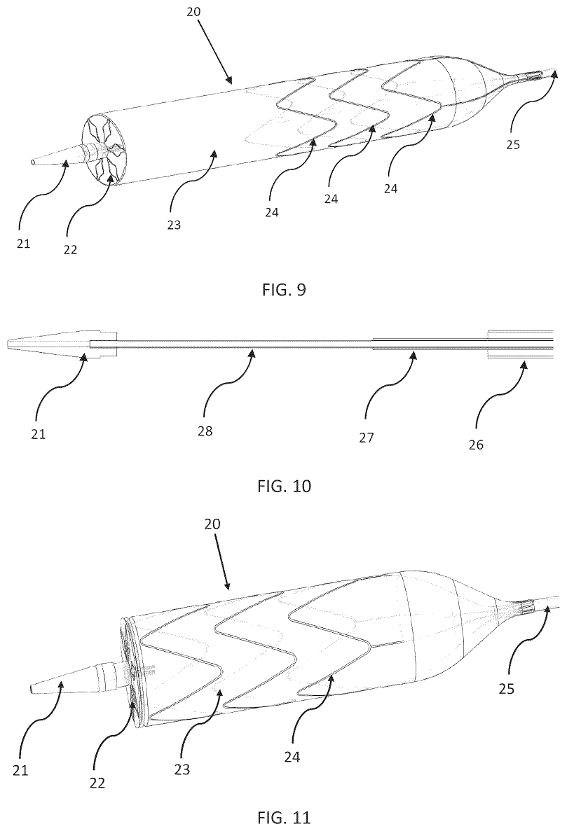

FIG. 9 illustrates device 20 fully deployed and free from outer shaft 26. In this figure, tip 21 which is connected to an inner shaft (not shown) is extended distally for the sake of clarity. Membrane 23 encompasses both radially expanding elements 24 and the radial plunger element 22. Radial expanding elements 24 are connected to an intermediate shaft (not shown). All shafts (inner, intermediate, and outer) are concentric. This is designed so that the inner shaft can move relative to the intermediate shaft which is stationary.

FIG. 10 illustrates the arrangement of the shafts of the embodiment, where outer shaft 26 can be moved to encompass device 20 and to radially compress the radially compressible elements 24 and membrane 23. Radially compressible elements 24 are fixed to intermediate shaft 27 and extend distally. Membrane 23 is connected at its distal end to radial plunger 22 which in turn is connected to inner shaft 28. Once outer shaft 26 is pulled back, radially compressible elements 24 open outward. When inner shaft 28 is pulled back, the distal end of membrane 23 which is connected to radial plunger 22 folds back onto itself and creates a vacuum.

FIG. 11 illustrates device 20 prior to inner tube 28 (not shown) being pulled back and causing the local vacuum.

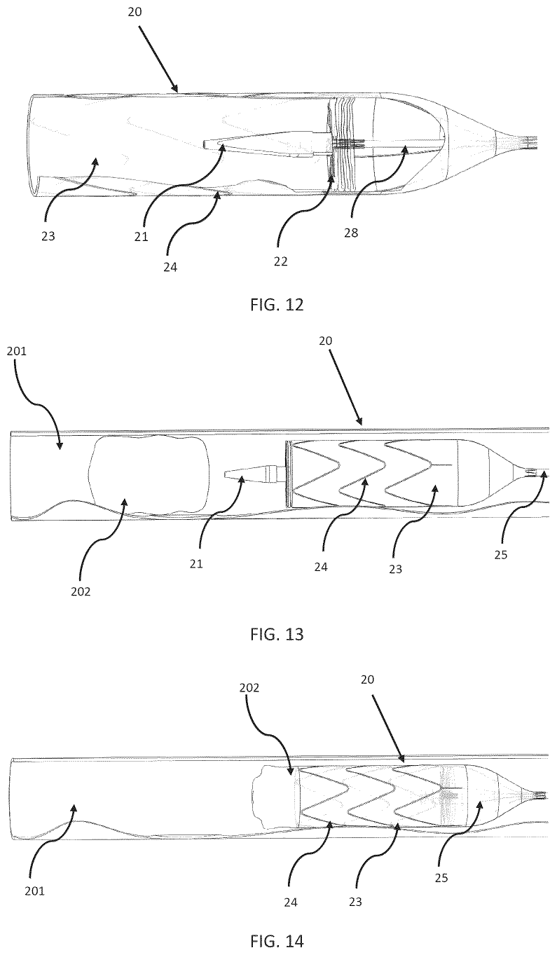

FIG. 12 illustrates device 20 with membrane 23 inverted inward once inner shaft 28 has been pulled back. Radial plunger 22 is shown in its rearward state and the folds of membrane 23 are visible.

FIG. 13 illustrates device 20 in an obstructed body lumen. The device is advanced towards the obstruction 202 and then the outer shaft 26 (not shown) is retracted thus expanding radially compressible elements 24 with membrane 23.

FIG. 14 illustrates the obstruction 202 “swallowed”, that is, pulled in, by device 20 due to the vacuum generated by pulling back inner shaft 28.

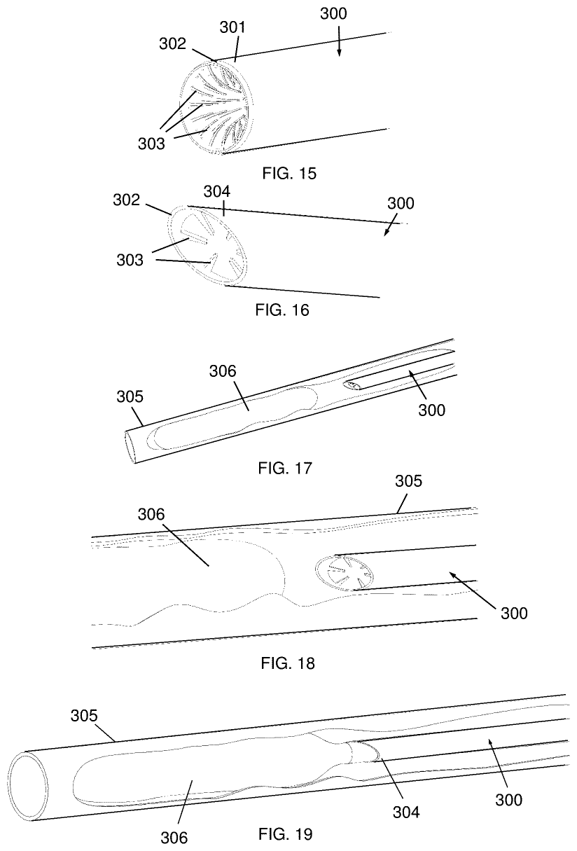

Reference is now made to FIG. 15, which illustrates a thrombus removal device, in accordance with another non-limiting embodiment of the invention. FIG. 15 shows a cannula 300 having at a distal end 301 thereof, a ring or plurality of rings 302. Each ring 302 includes a series of inwardly extending barbs 303 designed to snag the thrombus and prevent its release back into the circulatory system during cannula extraction in the event where vacuum is lost. The barbs 303 may have a backward or a forward orientation relative to the longitudinal axis of the cannula 300. The cannula 300 may have a blunt end, or may be cut at a chamfer to facilitate clot engagement. FIG. 15 illustrates a blunt cannula showing the toothed rings having a plurality of inwardly directional barbs which are oriented towards the distal end of the catheter.

FIG. 16 illustrates cannula 300 with a chamfered distal end 304, showing the toothed rings 302 with a plurality of inwardly directional barbs 303 which are oriented towards the distal end of the catheter.

FIG. 17 illustrates insertion of chamfered cannula 300 into an obstructed blood vessel 305 in which a thrombus 306 is located.

FIG. 18 illustrates cannula 300 advanced up to the thrombus site.

FIG. 19 illustrates the thrombus 306 aspirated, and it remains on the distal end 304 of the cannula 300. FIG. 19A is cutaway illustration showing how the thrombus 306 gets snagged on the barbs 303 of the toothed ring 302.

PCT patent application PCT/US2021/042676 (published as WO 2022/020539) describes another obstruction capture device. The structure of that obstruction capture device included a conical cone with grasper arms disposed at its distal end. The grasper arms are closed radially inwards against the obstruction so that the obstruction is pulled into the conical mesh. A risk associated with this type of device is the insertion of a relatively large captured clot into an aspiration cannula that has a smaller diameter than the obstruction. Once the capture clot is pulled into a cannula having a smaller diameter than the size of the clot, parts of the clot may be torn away and return to block the blood vessel.

The present invention provides solutions to this problem as is now described.

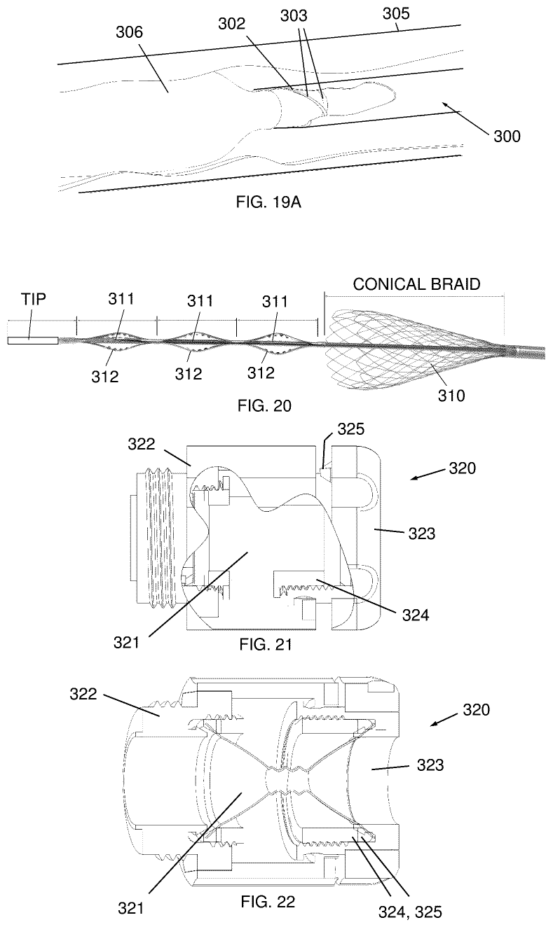

Reference is now made to FIG. 20, which illustrates a thrombus removal device, in accordance with another non-limiting embodiment of the invention. FIG. 20 illustrates a self-expanding conical mesh 310 located just proximal to capture (grasper) elements 311. A mesh cone 312 (which may be constructed like a stent mesh) is added over the capture elements 311 (or just proximal to the elements) such that once capture is achieved, the capture elements 311 with the captive clot are pulled into the mesh cone 312. This achieves two main objectives: it strengthens the capture efficiency by allowing an outer tube to be advanced over a section of the mesh cone 312, compressing the cone 312 radially inwards, and further capturing the proximal end of the clot. Secondly, the insertion of the captured clot into the cannula is enhanced by deforming the shape of the clot and preventing clot shear by the cannula wall since the clot is captured within the mesh cone 312.

During a typical procedure, a physician may attempt to pass therapeutic devices through a cannula having a proximal end outside the body and a distal end at a determined target site. The devices passed through the cannula may have differing diameters which range in size such as guidewires and dilators. During the interchange, excessive blood loss may occur. Typically, gaskets within the proximal end of the cannulas are used to limit blood loss; however, these gaskets have a limited size range and do not provide adequate sealing when the size range is large. In addition, in the case of aspiration cannulas, the gasket acts not only as a blood loss prevention element, but also as a seal to prevent loss of vacuum strength when negative pressure is applied. A seal which allows for a wide diameter variation and also as an effective vacuum seal is needed.

Reference is now made to FIGS. 21 and 22, which illustrate an adjustable seal valve 320 which can be attached to a proximal end of a cannula (not shown). The seal 320 has a deformable internal sleeve or tube 321 (shown in FIG. 22) captured at both ends, where a first end 322 is fixed and a second end 323 is free to rotate relative the first end 322. A series of radially placed indentations 324 on the first end 322 accept a protrusion 325 placed on the second rotating end 323 so as to prevent rotation of the second end 323 by locking the second end 323 into the first end 322. When the second rotatable end 323 is rotated, the deformable internal sleeve 321 deforms and collapses inwardly in a radial direction so that it effectively forms a seal around a therapeutic device of any diameter passed through it. The range of sealing diameters can be from 0.5 mm to 30 mm.

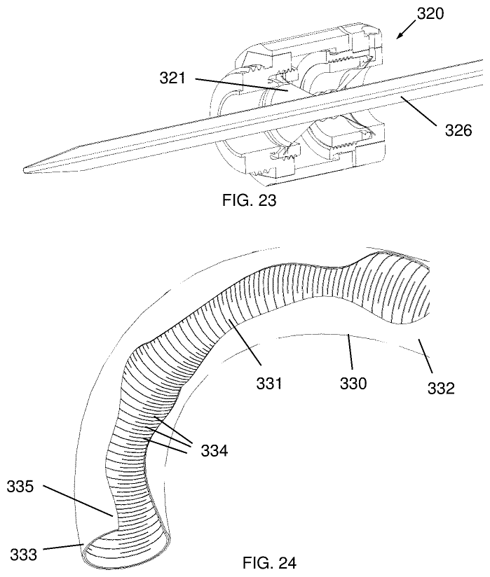

FIG. 23 illustrates the adjustable valve 320 with the inner tube 321 deformed to an intermediate level to seal over a therapeutic tool 326. The adjustable seal valve 320 seals equally well in high and low pressure situations (vacuum and positive pressures), and can seal against small diameter objects (such as a guidewire) and large diameter objects (such as dilators).

FIGS. 32A and 32B illustrate another version of the embodiments of FIGS. 21, 22 and 23. In this version, a spring 327 is coupled between the second rotatable end 323 (also called rotating collar 323) and a portion of the body of valve 320 which is coupled to a rotating knob 328. In the normal non-tensioned state of spring 327, the deformable internal sleeve 321 is in its twisted (closed) position. When the rotating knob 328 is turned (such as being moved within a rotation groove 329 formed in the valve housing), the rotating collar 323 moves the sleeve 321 to its open position. The amount of sleeve opening may be controlled by the amount of rotation of knob 328. The rotation of knob 328 causes tensioning of spring 327. When the rotating knob 328 is released, the tension on spring 327 is relaxed so that spring 327 provides a biasing force on the second rotatable end 323 to close second rotatable end 323 (either fully or partially, depending on how much the knob releases tension on the spring). Spring 327 may be any kind of suitable biasing device, such as but not limited to, a coil spring, leaf spring, torsion spring, flexible or elastomeric element, and others. The spring 327 is directly connected to the rotating hub (that is, second rotatable end 323), which ensures that the rotational movement of the rotating hub is positively and directly limited by the spring, which prevents the possibility of any jamming.

Flexibility of aspiration cannulas in general is an important issue for physicians. There are many tradeoffs when it comes to flexibility which may impede proper function of the cannula. In order to increase flexibility, manufacturers may sacrifice important aspects of the cannula such as compressibility, trackability and strength.

Reference is now made to FIG. 24, which illustrates a flexible cannula 330 constructed by combining an internal tube 331, also called hypotube 331, (which may be made of steel or a steel alloy or any other suitable stiff material) having a proximal end 332 and a distal end 333 and a series of slits 334 cut along its length. The slits 334 enable the tube 331 to bend in any direction by the expansion and compression of the slits 334. An outer softer jacket 335 which is partially connected to the inner tube 331. The outer softer jacket 335 may be made of a meltable polymer, such as but not limited to, nylon, polyether block amide (PEBA), and other materials. Examples of PEBA include the tradenames PEBAX (from Arkema) and VESTAMID (from Evonik Industries). Discrete portions of outer jacket 335 may be joined to inner tube 331 (such as by melting the polymer so that it sticks to the outside of inner tube 331) at discrete locations along the longitudinal axis of cannula 330. The areas at which there is no connection between outer jacket 335 and inner tube 331 allow some relative movement between the jacket 335 and the hypotube 331, resulting in increased flexibility of cannula 330.

FIG. 24 illustrates cannula 330 having slits 334 along its longitudinal length and having outer jacket 335, which may be made of a variety of polymers.

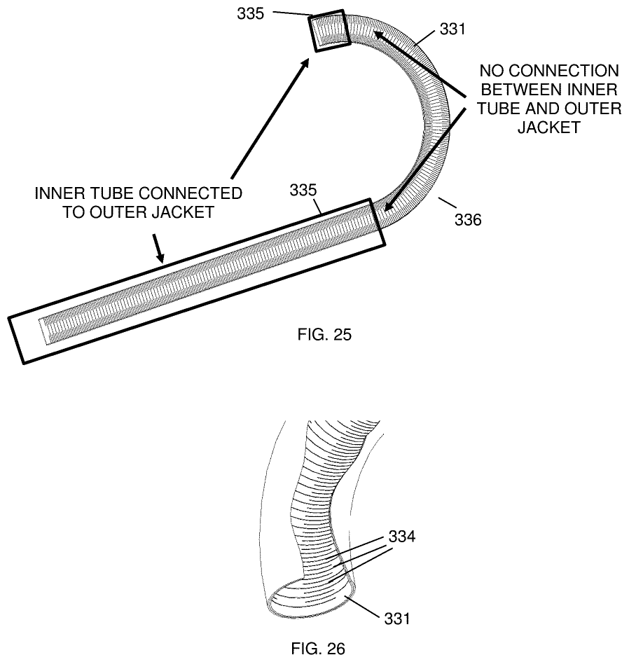

FIG. 25 illustrates gaps 336 in the connection zones between the inner tube 331 and the outer jacket 335.

FIG. 26 illustrates cut slits 334 along the hypotube may also serve to decrease the wall friction between the fluid flowing through the inner tube 331 (e.g., blood and clot material) and inner wall of tube 331 by a reduction of the Reynolds number.

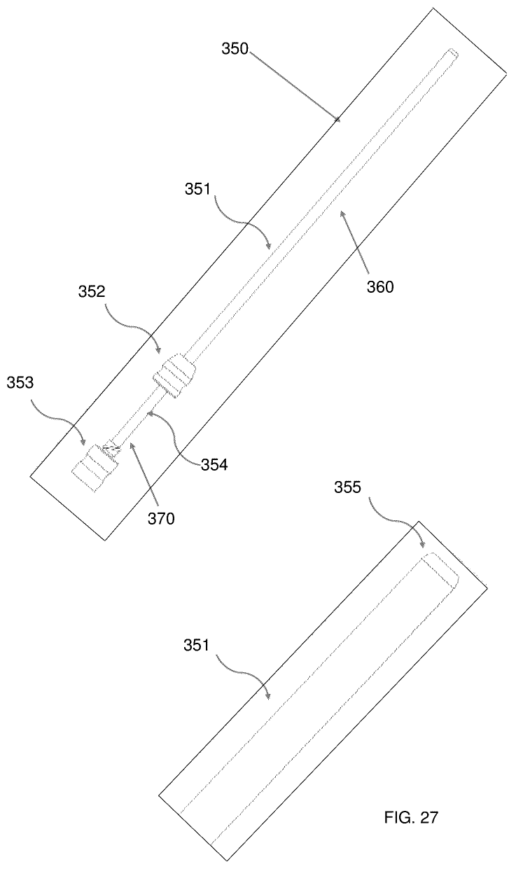

Reference is now made to FIG. 27, which illustrates a dual introducer sheath 350 which may be used during percutaneous procedures, in accordance with an embodiment of the invention. The sheath 350 includes two concentric sheaths-an inner sheath 370 and an outer sheath 360. Both sheaths consist of a shaft and a hemostatic hub, such that inner sheath 370 has a shaft 354 and a hub 353. Similarly, outer sheath 360 has a shaft 351 and a hub 352. A dilator (not shown) may be used to facilitate blood vessel entry as is known in the art. The shaft hubs are designed to provide hemostatic sealing. The distal end of outer shaft 351 has a chamfer 355 to prevent vessel damage during insertion.

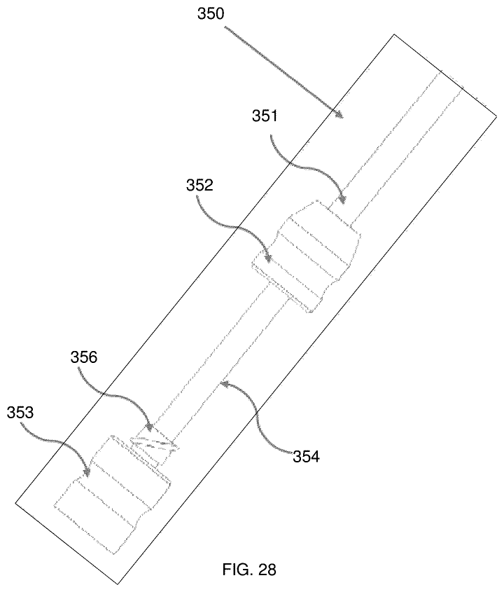

FIG. 28 illustrates dual sheath device 350 showing the orientation of the shafts and hubs prior to insertion into the blood vessel. Inner hub 353 may have a connection section 356 which may be used to connect inner hub 353 to outer hub 352.

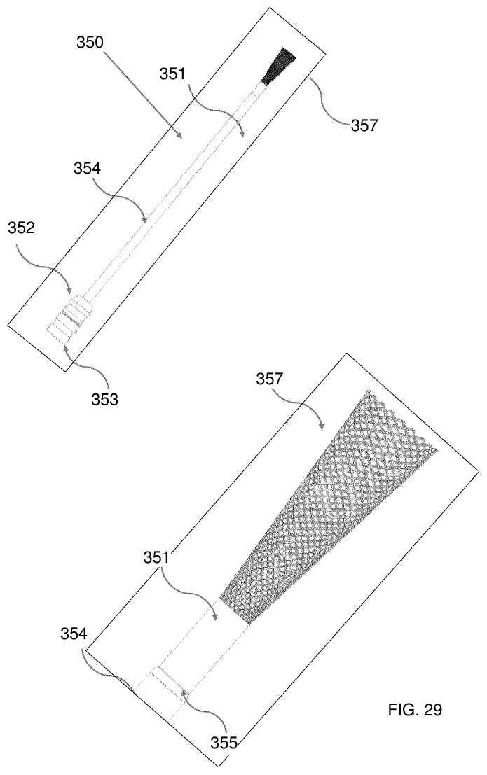

FIG. 29 illustrates the outer sheath 360 as it is pulled back relative to inner sheath 370 so that hubs 352 and 353 are connected. When outer sheath 360 is in its retracted position, a cone mesh 357 which is connected to inner shaft 354 is exposed.

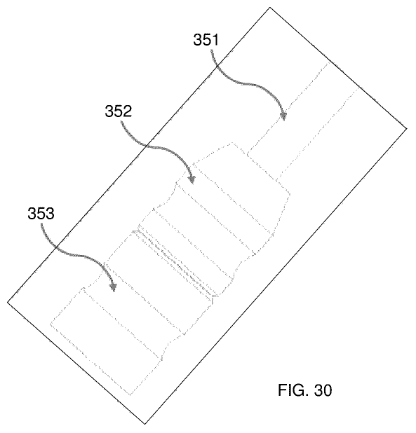

FIG. 30 illustrates hubs 352 and 353 in their locked position.

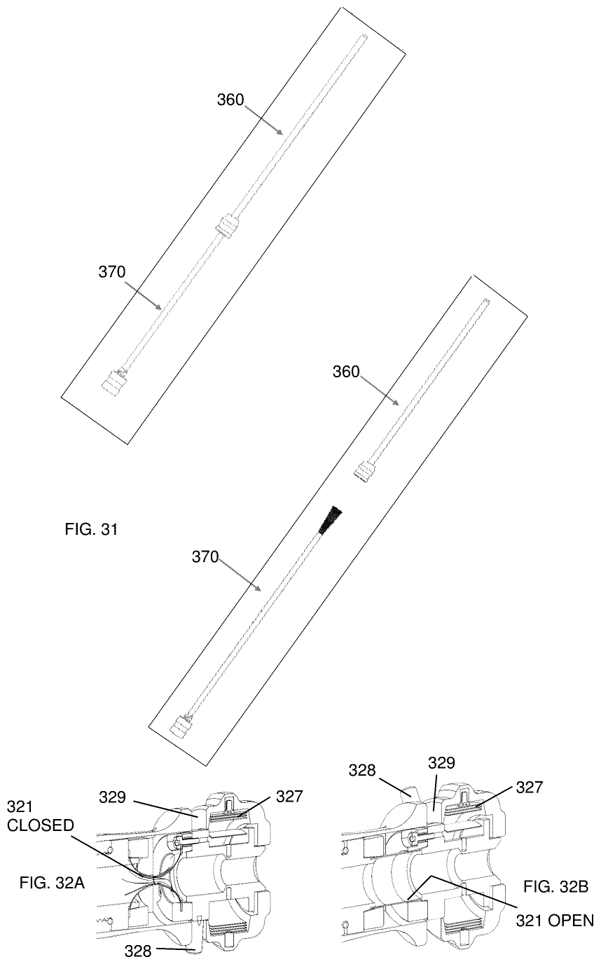

FIG. 31 illustrates inner sheath 370 as it is removed from outer sheath 360.

The dual sheath system may be used to remove large obstructions from the vasculature. Any therapeutic device used to capture and remove obstructions from the vasculature may be passed through the dual sheath system 350. Should the obstructions be larger than the diameter of the sheath system, the therapeutic device may be pulled back to a position where the obstruction is located within cone mesh 357. At this point, inner sheath 370 may be removed from outer sheath 360, safely removing the obstruction from the body. With this dual sheath system, the obstruction may be removed while keeping a protective sheath in place (the outer sheath remains within the vasculature). This is different from the prior art in which a single sheath is used and therefore mandates a vascular surgical procedure to be performed when removing a large obstruction.

It is noted that any embodiment described herein may be combined with one or more other embodiments described herein.

1. A medical device comprising:
a flexible cannula that comprises an internal tube having a proximal end and a distal end and a series of slits cut along a length thereof between said proximal and said distal ends; and
an outer jacket softer than said inner tube, wherein discrete portions of said outer tube are connected to said inner tube along a longitudinal axis of said cannula, and areas at which there is no connection between said outer jacket and said inner tube allow some relative movement between said outer jacket and said inner tube, resulting in increased flexibility of said cannula.

2. The medical device according to claim 1, wherein said outer jacket is made of a meltable polymer, and said discrete portions of said outer jacket comprise melted portions that stick to an outside of said inner tube.

3. The medical device according to claim 1, wherein said slits enabling said internal tube to bend in any direction by expansion and compression of said slits.

4. The medical device according to claim 1, wherein said medical device further comprises:
a thrombus removal device that comprises a distal removal element, coupled at a distal portion thereof to an inner shaft, and located within an outer shaft configured to guide the distal removal element towards a target site, wherein said distal removal element comprises radial elements which are exposed by retracting said outer shaft, and wherein said distal removal element is opened by pulling said inner shaft relative to an intermediate shaft, thereby compressing said distal removal element and forcing said radial elements to open radially outward to an open position.

5. The medical device according to claim 4, wherein a membrane covers said radial elements, and pulling said inner shaft causes a distal end of said membrane, which is connected to a radial plunger, to fold back onto itself and create a vacuum.

6. The medical device according to claim 5, wherein said membrane is a blood-impervious membrane.

7. The medical device according to claim 5, wherein said inner shaft, said intermediate shaft and said outer shaft are concentric with each other.

8. The medical device according to claim 4, wherein some of said radial arms have larger radial diameters in the open position than others of said radial arms.

9. The medical device according to claim 4, wherein some of said radial arms have different sinusoidal or other wavy shapes than others of said radial arms.

10. The medical device according to claim 1, wherein an adjustable seal valve is coupled to said flexible cannula, said adjustable seal valve comprising a first end which is fixed and a second end which is free to rotate relative said first end, and wherein a deformable internal sleeve is coupled to said first end and to said second end, and wherein radially placed indentations on said first end are configured to receive a protrusion placed on said second end so as to lock said second end into said first end and prevent rotation of said second end, and wherein rotation of said second end causes said deformable internal sleeve to deform and collapse inwardly in a radial direction so as to form a seal around an object of any diameter passing through said deformable internal sleeve.

11. A medical device comprising:
a thrombus removal device that comprises a distal removal element, coupled at a distal portion thereof to an inner shaft, and located within an outer shaft configured to guide the distal removal element towards a target site, wherein said distal removal element comprises radial elements which are exposed by retracting said outer shaft, and wherein said distal removal element is opened by pulling said inner shaft relative to an intermediate shaft, thereby compressing said distal removal element and forcing said radial elements to open radially outward to an open position.

12. The medical device according to claim 11, wherein a membrane covers said radial elements, and pulling said inner shaft causes a distal end of said membrane, which is connected to a radial plunger, to fold back onto itself and create a vacuum.

13. The medical device according to claim 11, wherein said inner shaft, said intermediate shaft and said outer shaft are concentric with each other.

14. The medical device according to claim 11, wherein some of said radial arms have larger radial diameters in the open position than others of said radial arms.

15. The medical device according to claim 11, wherein some of said radial arms have different sinusoidal or other wavy shapes than others of said radial arms.
